(19) United States
(12) Patent Application Publication
(10) Pub. No.: US 2017/0196442 A1
(43) Pub. Date: Jul. 13, 2017

(54) CAPSULE ENDOSCOPE SYSTEM

(71) Applicant: OLYMPUS CORPORATION, Tokyo (JP)

(72) Inventor: Daisuke SATO, Tokyo (JP)

(73) Assignee: OLYMPUS CORPORATION, Tokyo (JP)

(21) Appl. No.: 15/468,269

(22) Filed: Mar. 24, 2017

Related U.S. Application Data
(63) Continuation of application No. PCT/JP2015/075872, filed on Sep. 11, 2015.

(30) Foreign Application Priority Data
Dec. 8, 2014 (JP) 2014-247893

(51) Int. Cl. A61B 1/04; A61B 1/06

(52) U.S. Cl. CPC A61B 1/041; A61B 1/06

(57) ABSTRACT

A system includes: a capsule endoscope configured to be introduced into a subject to generate an image signal, and a control device for generating an image of the subject. The capsule endoscope includes: a first transmitter for sending information indicating a posture of the capsule endoscope; a first receiver for receiving a control signal from the control device; and a changing mechanism for changing the posture of the capsule endoscope based on the control signal. The control device includes: a second receiver for acquiring the information indicating the posture; a detector for detecting the posture of the capsule endoscope based on the information indicating the posture; a calculator for calculating a target posture of the capsule endoscope based on the image; and a signal generator for generating the control signal based on a detection result of the posture of the capsule endoscope and on the target posture.

### CROSS-REFERENCE TO RELATED APPLICATIONS

This application is a continuation of PCT International Application No. PCT/JP2015/075872, filed on Sep. 11, 2015 which designates the United States, incorporated herein by reference, and which claims the benefit of priority from Japanese Patent Application No. 2014-247893, filed on Dec. 8, 2014, incorporated herein by reference.

### BACKGROUND

1. Technical Field

The disclosure relates to a capsule endoscope system having a capsule endoscope configured to be introduced into a subject to capture images.

2. Related Art

In recent years, in the field of endoscopes, there has been known capsule endoscopes configured to be introduced into a subject to capture images. The capsule endoscopes have an imaging function and a wireless communication function inside a capsule-shaped casing small enough to be introduced into a lumen (digestive canal) of the subject. The capsule endoscope is swallowed by the subject and thereafter captures images while being moved in the lumen by peristaltic action of the lumen and sequentially wirelessly transmits image data. The wirelessly transmitted image data is received by a receiving device provided outside the subject. Further, the image data is taken into an image display device such as a workstation and predetermined image processing is performed on the image data. It is therefore possible to display an image of inside of the subject as a still image or a moving image (for example, see JP 2009-247494 A).

### SUMMARY

In some embodiments, a capsule endoscope system includes a capsule endoscope configured to be introduced into a subject, configured to perform imaging to generate an image signal, and configured to wirelessly transmit the image signal, and includes a control device configured to receive the image signal to generate an image of an inside of the subject based on the image signal. The capsule endoscope includes: a first transmitter configured to send information indicating a posture of the capsule endoscope; a first receiver configured to receive a signal transmitted from the control device; and a posture changing mechanism configured to change the posture of the capsule endoscope. The control device includes: a second receiver configured to acquire the information indicating the posture sent by the first transmitter; a posture detector configured to detect the posture of the capsule endoscope based on the information indicating the posture; a target posture calculator configured to calculate a target posture of the capsule endoscope based on the image; a posture control signal generator configured to generate a control signal for changing the posture of the capsule endoscope based on a detection result of the posture of the capsule endoscope and based on the target posture; and a second transmitter configured to transmit the control signal to the capsule endoscope. The first receiver is configured to receive the control signal transmitted by the second transmitter, and the posture changing mechanism is configured to change the posture of the capsule endoscope based on the control signal received by the first receiver.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram illustrating a configuration example of a capsule endoscope system according to a first embodiment of the present invention;

FIG. 2 is a schematic diagram illustrating an appearance of the capsule endoscope system illustrated in FIG. 1;

FIG. 3 is a schematic diagram illustrating an example of an internal structure of a capsule endoscope illustrated in FIG. 1;

FIGS. 4A and 4B are schematic diagrams illustrating a configuration example of a posture changing unit illustrated in FIG. 1;

FIG. 5 is a schematic diagram for explaining variables representing a posture of the capsule endoscope illustrated in FIG. 1;

FIG. 6 is a flowchart illustrating an operation of the capsule endoscope illustrated in FIG. 1;

FIG. 7 is a flowchart illustrating an operation of a control device illustrated in FIG. 1;

FIG. 8 is a schematic diagram illustrating a state in which the capsule endoscope moves in a lumen of the subject;

FIG. 9 is a schematic diagram illustrating an image captured from a visual field of the capsule endoscope illustrated in FIG. 8;

FIG. 10 is a schematic diagram illustrating a state in which the capsule endoscope moves in a lumen of the subject;

FIG. 11 is a schematic diagram illustrating an image captured from a visual field of the capsule endoscope illustrated in FIG. 10;

FIGS. 12A and 12B are schematic diagrams illustrating a configuration example of a posture changing unit included in a capsule endoscope according to a first modified example of the first embodiment of the present invention;

FIGS. 13A and 13B are schematic diagrams illustrating a configuration example of a posture changing unit included in a capsule endoscope according to a second modified example of the first embodiment of the present invention;

FIG. 14 is a block diagram illustrating a configuration of a capsule endoscope system according to a second embodiment of the present invention;

FIG. 15 is a block diagram illustrating a configuration of a capsule endoscope system according to a third embodiment of the present invention; and

FIG. 16 is a flowchart illustrating an operation of a control device illustrated in FIG. 15.

### DETAILED DESCRIPTION

Hereinafter, a capsule endoscope system according to embodiments of the present invention will be described with reference the drawings. In the description below, each drawing merely schematically illustrates shapes, sizes, and positional relationships in a degree such that contents of the present invention can be understood. Therefore, the present invention is not limited to the sizes, the shapes, and the positional relationships illustrated in each drawing. The same reference signs are used to designate the same elements throughout the drawings.

### First Embodiment

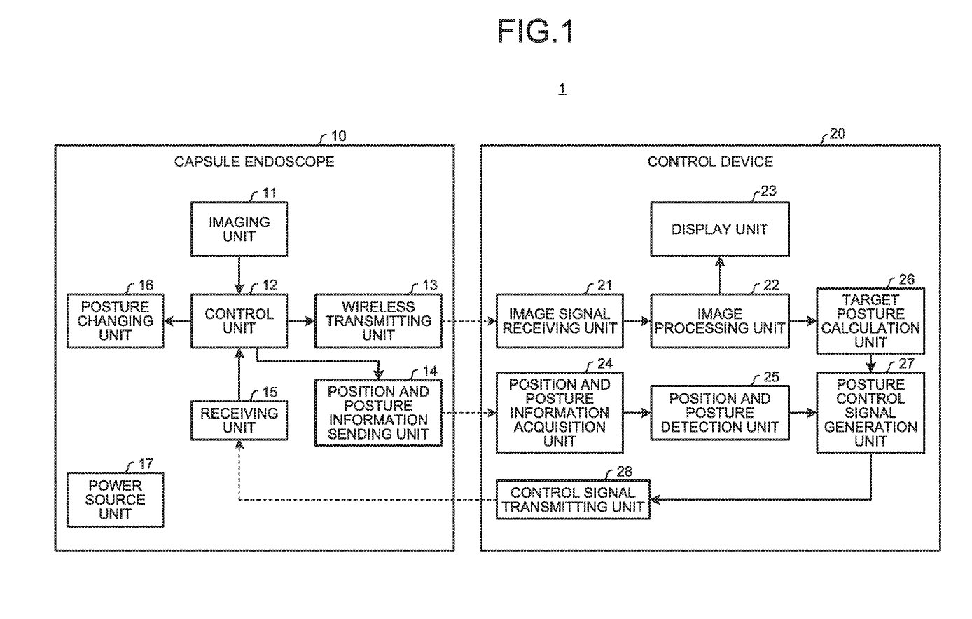

FIG. 1 is a block diagram illustrating a configuration example of a capsule endoscope system according to a first embodiment of the present invention. As illustrated in FIG. 1, a capsule endoscope system 1 according to the first embodiment includes a capsule endoscope 10 that is configured to be introduced into a subject, captures images, and generates an image signal, and a control device 20 that generates an in-vivo image based on the image signal generated by the capsule endoscope 10.

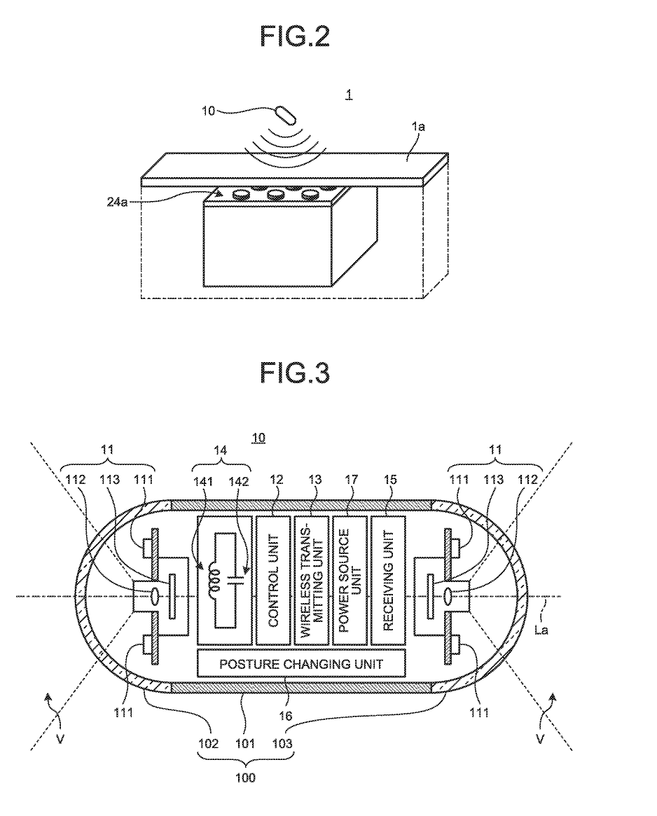

FIG. 2 is a schematic diagram illustrating an appearance of the capsule endoscope system 1. As illustrated in FIG. 2, the capsule endoscope system 1 is provided with a bed 1a on which a subject is mounted.

The capsule endoscope 10 is configured to be introduced into the subject by, for example, oral ingestion, and thereafter moves in a lumen (digestive canal) of the subject, and is finally discharged to the outside of the subject. During that time, the capsule endoscope 10 captures images of the inside of an organ of the subject, generates image signals, and sequentially wirelessly transmits the image signals to the outside of the subject.

FIG. 3 is a schematic diagram illustrating an example of an internal structure of the capsule endoscope 10. As illustrated in FIG. 3, the capsule endoscope 10 includes a capsule-shaped casing 100 that is an outer casing small enough to be easily introduced into an organ of the subject, two imaging units 11 that capture images of the subject in directions different from each other, a control unit 12 that processes a signal input from each imaging unit 11 and controls each element of the capsule endoscope 10, a wireless transmitting unit 13 that transmits the signal processed by the control unit 12 to the outside of the capsule endoscope 10, a position and posture information sending unit 14 that sends information indicating position and posture of the capsule endoscope 10, a receiving unit 15 that receives a control signal wirelessly transmitted from the outside, a posture changing unit 16 that changes the posture of the capsule endoscope 10, and a power source unit 17 that supplies power to each element of the capsule endoscope 10.

The capsule-shaped casing 100 includes a tubular casing 101 and dome-shaped casings 102 and 103 and is formed by closing open ends of both sides of the tubular casing 101 with the dome-shaped casings 102 and 103. The tubular casing 101 is a colored casing that is substantially opaque to visible light. On the other hand, the dome-shaped casings 102 and 103 are dome-shaped optical members that are transparent to light of a predetermined wavelength band such as visible light. The capsule-shaped casing 100 internally includes the imaging unit 11, the control unit 12, the wireless transmitting unit 13, the position and posture information sending unit 14, the receiving unit 15, the posture changing unit 16, and the power source unit 17 in a liquid-tight manner.

Each imaging unit 11 has an illumination unit 111 which is formed of an LED (Light Emitting Diode), an LD (Laser Diode), or the like and emits illumination light such as white light, an optical system 112 such as a condenser lens, and an image sensor 113 formed of a CMOS image sensor, a CCD, or the like. The illumination unit 111 emits illumination light to the subject in a visual field V of each image sensor 113 through the dome-shaped casing 102 or 103. The optical system 112 condenses reflection light from the visual field V and forms an image on an imaging surface of the image sensor 113. The image sensor 113 converts the reflection light (optical signal) from the visual field V, which is received on the imaging surface, into an electrical signal and outputs the electrical signal as an image signal.

The two imaging units 11 are arranged such that optical axes of the optical systems 112 of the imaging units 11 are substantially in parallel with or substantially coincident with a long axis La which is a center axis in a longer direction of the capsule-shaped casing 100 and the visual fields V of the two imaging units 11 face directions opposite to each other. In other words, the two imaging units 11 are mounted such that the imaging surface of each image sensor 113 is perpendicular to the long axis La.

The first embodiment employs a multiple-camera capsule endoscope in which the two imaging units 11 capture images, respectively, in both directions (front and rear directions) of the long axis La of the capsule endoscope 10. However, a single-camera capsule endoscope may be employed in which only one imaging unit 11 is provided to capture images in one direction of the long axis La.

The control unit 12 controls each operation of the imaging unit 11, the wireless transmitting unit 13, the position and posture information sending unit 14, the receiving unit 15, and the posture changing unit 16 and controls input and output of signals between these units. Further, the control unit 12 sets an imaging frame rate of the imaging unit 11, causes the image sensor 113 to capture images of the subject in the visual field V illuminated by the illumination unit 111, and performs predetermined signal processing on the image signal output from the image sensor 113.

The wireless transmitting unit 13 includes an antenna (not illustrated in the drawings) for transmitting a wireless signal. The wireless transmitting unit 13 acquires the image signal on which the signal processing is performed by the control unit 12, generates a wireless signal by performing modulation processing and the like on the image signal, and transmits the wireless signal to the control device 20 through the antenna.

The position and posture information sending unit 14 (first transmitter) includes a coil 141 that forms a part of a resonance circuit and generates a magnetic field by receiving a power supply and a capacitor 142 that forms the resonance circuit along with the coil 141. The position and posture information sending unit 14 generates a magnetic field of a predetermined frequency by receiving a power supply from the power source unit 17 under control of the control unit 12. In the first embodiment, the magnetic field is used as information that represents the position and posture.

The receiving unit 15 (first receiver) is a control signal receiving unit that receives various control signals wirelessly transmitted from the control device 20 and outputs the control signals to the control unit 12. Specifically, the control signals include a posture control signal for changing the posture of the capsule endoscope 10.

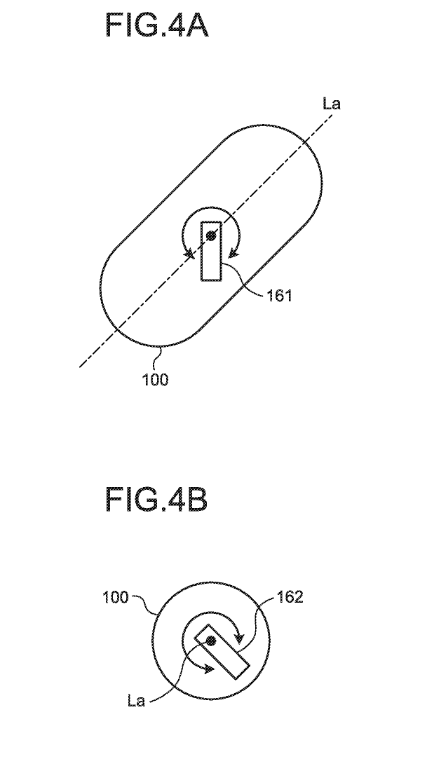

The posture changing unit 16 (posture changing mechanism) changes the posture of the capsule endoscope 10 according to the posture control signal received by the receiving unit 15 under control of the control unit 12. FIGS. 4A and 4B are schematic diagrams illustrating a configuration example of the posture changing unit 16. FIG. 4A illustrates the capsule-shaped casing 100 as viewed from a side surface and FIG. 4B illustrates the capsule-shaped casing 100 as viewed from the long axis La direction.

As illustrated in FIGS. 4A and 4B, the posture changing unit 16 includes an eccentric motor 161 that rotates in a plane containing the long axis La of the capsule-shaped casing 100, an eccentric motor 162 that rotates around the long axis La, and a drive unit (not illustrated in the drawings) that drives each of the eccentric motors 161 and 162. The posture changing unit 16 rotates the eccentric motors 161 and 162 by a predetermined angle under control of the control unit 12 based on the posture control signal wirelessly transmitted from the control device 20. In this way, the position of the center of gravity of the capsule endoscope 10 changes and the posture of the capsule endoscope 10 is changed.

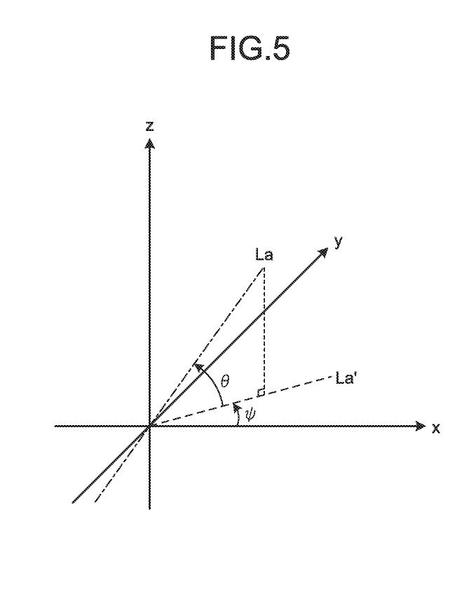

Here, the posture of the capsule endoscope 10 can be represented by various variables. FIG. 5 is a schematic diagram for explaining variables representing the posture of the capsule endoscope 10 in the first embodiment. As illustrated in FIG. 5, in the first embodiment, the posture of the capsule endoscope 10 is represented by an angle (elevation angle) A of the long axis La of the capsule endoscope 10 with respect to the horizontal plane (xy plane) and an angle (turning angle) ψ of the long axis La around the vertical axis (z axis). The turning angle ψ is a rotation angle of an axis La′, which is obtained by projecting the long axis La onto the xy plane, from the x axis.

The power source unit 17 is a power storage unit such as a button-type battery or a capacitor and has a switch unit such as a magnetic switch or an optical switch. When the power source unit 17 is configured to have a magnetic switch, the power source unit 17 switches between ON and OFF of a power source by a magnetic field applied from outside. When the power source is in an ON state, the power source unit 17 supplies power of the power storage unit to each element (the imaging unit 11, the control unit 12, and the wireless transmitting unit 13, the position and posture information sending unit 14, the receiving unit 15, and the posture changing unit 16) of the capsule endoscope 10. When the power source is in an OFF state, the power source unit 17 stops power supply to each element of the capsule endoscope 10.

Referring to FIG. 1 again, the control device 20 includes an image signal receiving unit 21 that receives an image signal wirelessly transmitted from the capsule endoscope 10, an image processing unit 22 that generates an image based on the image signal received by the image signal receiving unit 21 and performs predetermined image processing on the image, a display unit 23 that displays an image and related information of the image, a position and posture information acquisition unit 24 that acquires information indicating the position and posture transmitted from the capsule endoscope 10, a position and posture detection unit 25 that detects current position and posture of the capsule endoscope 10 based on the information indicating the position and posture received by the position and posture information acquisition unit 24, a target posture calculation unit 26 that calculates a target posture of the capsule endoscope 10, a posture control signal generation unit 27 that generates a posture control signal for changing the posture of the capsule endoscope 10, and a control signal transmitting unit 28 that wirelessly transmits the posture control signal to the capsule endoscope 10.

The image signal receiving unit 21 includes a plurality of receiving antennas and sequentially receives wireless signals transmitted by the capsule endoscope 10 through the receiving antennas. The plurality of antennas is arranged on a body surface of the subject and used. The image signal receiving unit 21 selects a receiving antenna whose reception electric field intensity is the highest from among the receiving antennas, extracts an image signal from a wireless signal received through the selected receiving antenna by performing demodulation processing on the wireless signal, and outputs the image signal to the image processing unit 22.

The image processing unit 22 generates display image data that represents an image of inside of the subject by performing image processing such as white balance processing, demosaicing, color conversion, density conversion (gamma conversion or the like), smoothing (noise removal and the like), and sharpening (edge enhancement and the like) on the image signal output from the image signal receiving unit 21.

The display unit 23 has a screen formed from any kind of display such as a liquid crystal display and displays an image based on the image data generated by the image processing unit 22, the position and posture of the capsule endoscope 10 detected by the position and posture detection unit 25 described later, and other various information on the screen.

The position and posture information acquisition unit 24 (second receiver) has a plurality of sensing coils 24a (see FIG. 2) that detects the magnetic field generated by the capsule endoscope 10. The plurality of sensing coils 24a are arranged on a planar panel arranged in parallel with the upper surface of the bed 1a. Each sensing coil 24a is formed from a cylindrical coil having, for example, a coil spring shape. The position and posture information acquisition unit 24 detects an electrical current generated in each sensing coil 24a by the effect of the magnetic field generated by the position and posture information sending unit 14 of the capsule endoscope 10.

The position and posture detection unit 25 (posture detector) receives a plurality of detection signals (electrical currents respectively generated in the plurality of sensing coils 24a) from the position and posture information acquisition unit 24 and extracts magnetic field information including the amplitude and phase of the magnetic field transmitted from the capsule endoscope 10 by performing signal processing such as waveform shaping, amplification, A/D conversion, and FFT on the detection signals. Further, the position and posture detection unit 25 calculates three-dimensional coordinates, the elevation angle θ, and the turning angle ψ (see FIG. 5) of the capsule endoscope 10 based on the magnetic field information, outputs the three-dimensional coordinates of the capsule endoscope 10 as the position information, and outputs the elevation angle θ and the turning angle ψ as the posture information.

The target posture calculation unit 26 (target posture calculator) calculates the target posture of the capsule endoscope 10 based on an image captured from the current visual field V of the capsule endoscope 10.

The posture control signal generation unit 27 (posture control signal generator) generates a posture control signal for changing the posture of the capsule endoscope 10 from the current posture to the target posture based on the current posture information of the capsule endoscope 10 output from the position and posture detection unit 25 and the target posture of the capsule endoscope 10 output from the target posture calculation unit 26.

The control signal transmitting unit 28 (second transmitter) wirelessly transmits the posture control signal generated by the posture control signal generation unit 27 to the capsule endoscope 10.

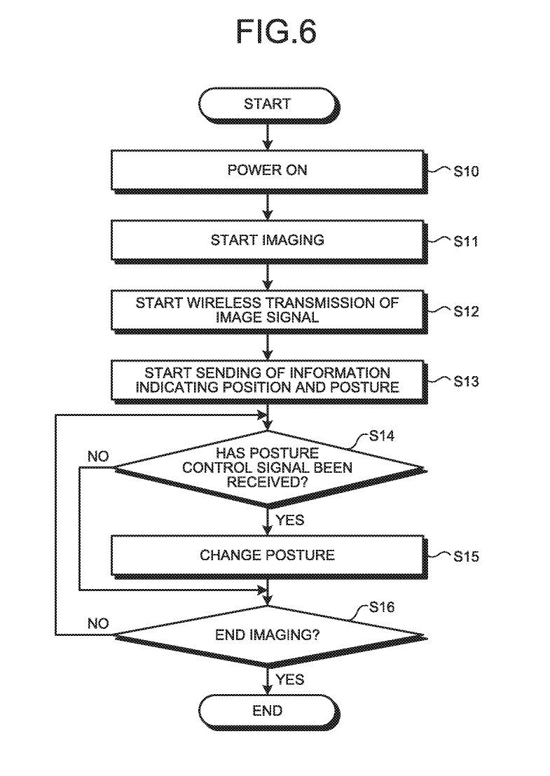

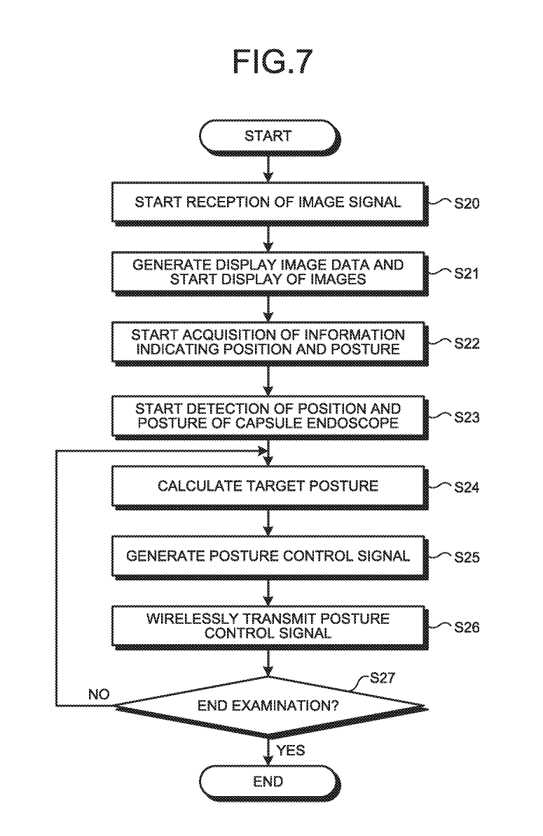

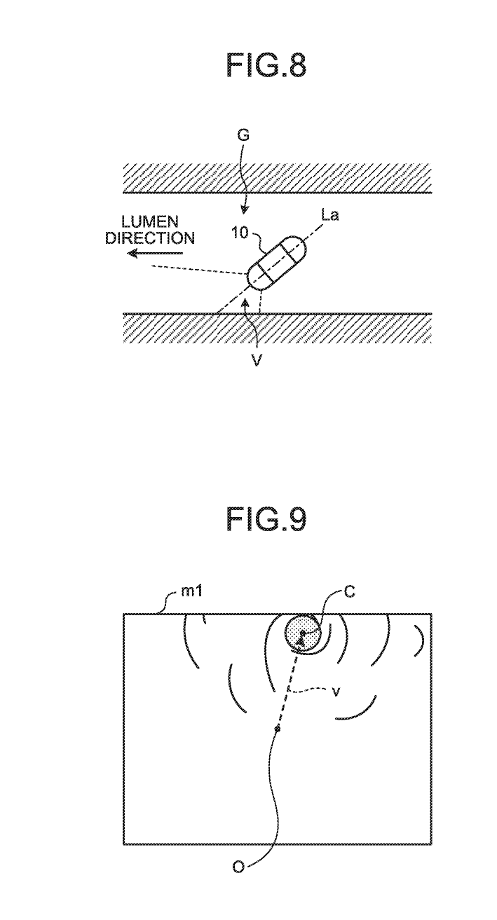

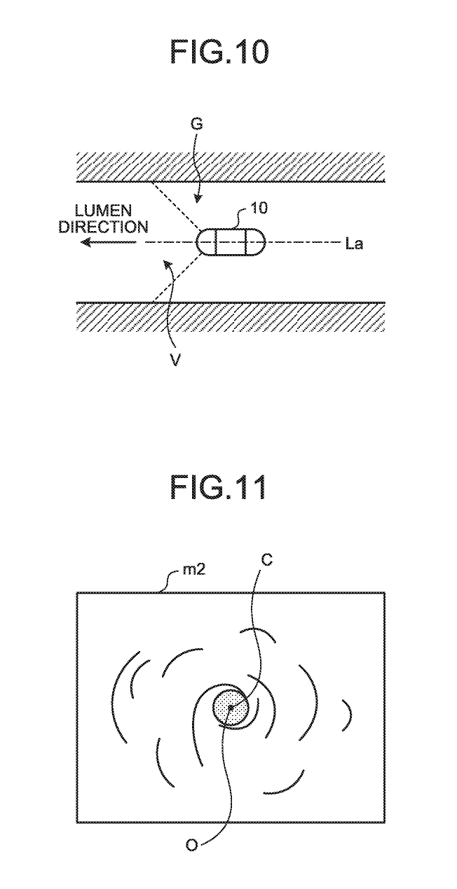

Next, an operation of the capsule endoscope system 1 will be described with reference to FIGS. 6 to 11. FIG. 6 is a flowchart illustrating an operation of the capsule endoscope 10. FIG. 7 is a flowchart illustrating an operation of the control device 20. FIGS. 8 and 10 are schematic diagrams illustrating a state in which the capsule endoscope 10 moves in a lumen of the subject. FIGS. 9 and 11 are schematic diagrams illustrating an image captured from a visual field of the capsule endoscope 10.

First, as illustrated in FIG. 6, in step S10, the capsule endoscope 10 is turned on. Then, in step S11, the imaging unit 11 starts imaging at a predetermined imaging frame rate.

In the following step S12, the wireless transmitting unit 13 starts wireless transmission of an image signal which is output from the imaging unit 11 and on which signal processing is performed by the control unit 12.

In step S13, the control unit 12 causes the position and posture information sending unit 14 to start sending of information indicating the position and posture. Specifically, the control unit 12 causes the power source unit 17 to start power supply to the position and posture information sending unit 14 and causes the position and posture information sending unit 14 to generate a magnetic field. In this case, it is preferable that the control unit 12 controls the power supply to the position and posture information sending unit 14 in synchronization with the imaging frame rate of the imaging unit 11, thereby causing the position and posture information sending unit 14 to generate a pulse signal of magnetic field.

Meanwhile, as illustrated in FIG. 7, in step S20, the image signal receiving unit 21 of the control device 20 starts reception of the image signal wirelessly transmitted from the capsule endoscope 10.

In the following step S21, the image processing unit 22 receives the image signal from the image signal receiving unit 21 and performs image processing such as white balance processing, demosaicing, color conversion, density conversion (gamma conversion or the like), smoothing (noise removal and the like), and sharpening (edge enhancement and the like) on the image signal. In this way, the image processing unit 22 generates display image data, outputs the image data to the display unit 23, and starts display of images of inside of the subject.

In step S22, the position and posture information acquisition unit 24 starts detection of the magnetic field generated by the capsule endoscope 10 as an acquisition operation of the position and posture information.

In step S23, the position and posture detection unit 25 receives a detection signal of the magnetic field from the position and posture information acquisition unit 24 and starts detection of the position and posture of the capsule endoscope 10 based on the detection signal.

At this stage, a user (a medical worker in charge of examination) confirms that the capsule endoscope 10 starts operation and then have the subject to swallow the capsule endoscope 10. Specifically, the user confirms whether the illumination unit 111 of the capsule endoscope 10 emits light periodically, whether the control device 20 receives the wireless signal transmitted from the capsule endoscope 10, or whether an image captured from a visual field of the capsule endoscope 10 is displayed on the display unit 23.

In step S24, from the image processing unit 22, the target posture calculation unit 26 receives image data based on an image signal wirelessly transmitted at a timing around (preferably, immediately before) transmission of information (generation of magnetic field) representing the position and posture that is used to detect the current position and posture of the capsule endoscope 10, and calculates the target posture of the capsule endoscope 10 based on an image corresponding to the image data.

Here, as illustrated in FIG. 8, when the long axis La of the capsule endoscope 10 is inclined with respect to a direction in which a lumen G extends (hereinafter referred to as a lumen direction) and a front visual field V of the capsule endoscope 10 faces downward as illustrated in FIG. 9, a central portion C in the lumen direction is shifted upward in an image m1. In this case, a part (an upper part in the case of FIG. 9) of peripheral region of the central portion C in the lumen direction is out of the image m1. Therefore, as illustrated in FIG. 10, it is preferable to control the posture of the capsule endoscope 10 such that the long axis La of the capsule endoscope 10 is in parallel with the lumen direction as much as possible.

Therefore, the target posture calculation unit 26 first detects the position of the central portion C in the lumen direction from the image m1. As a detection method of the central portion C in the lumen direction, it is possible to apply various known methods. As an example, the target posture calculation unit 26 estimates a distance between the capsule endoscope 10 and the subject (a mucosal surface inside the lumen G) shown in the image m1 based on pixel values of pixels in the image m1 and defines that a point where the distance is the greatest is the central portion C in the lumen direction. The distance between the capsule endoscope 10 and the subject can be estimated from R value of pixel values (R value, G value, and B value) and the brightness of each pixel in the image m1. Here, since a red component (R component) of the illumination light (white light) emitted from the capsule endoscope 10 is farthest away from an absorption band of blood and is a longest wavelength component, the red component is less likely to be affected by absorption or scattering in a living body. Therefore, the intensity of the R component best reflects the length of an optical path of the illumination light which is emitted from the capsule endoscope 10, is reflected by the subject, and enters the capsule endoscope 10. Specifically, the longer the distance between the capsule endoscope 10 and the subject, the smaller the R value and the brightness.

Then, the target posture calculation unit 26 calculates a directional vector v directed from a center point O of the image m1 corresponding to the center of the visual field V of the capsule endoscope 10 to the central portion C in the lumen direction on the image m1. Further, the target posture calculation unit 26 calculates a posture where the center of the visual field V of the capsule endoscope 10 faces the lumen direction as the target posture of the capsule endoscope 10 from the length (the number of pixels in the image m1) and the orientation of the directional vector v.

In the following step S25, the posture control signal generation unit 27 generates a posture control signal for changing the posture of the capsule endoscope 10 based on the current posture of the capsule endoscope 10 detected in step S23 and the target posture calculated in step S24. Specifically, the posture control signal generation unit 27 calculates a posture (elevation angle θ+Δθ, turning angle ψ+Δψ) where the target posture is added to the current posture of the capsule endoscope 10 (elevation angle θ and turning angle ψ, see FIG. 5). Then, the posture control signal generation unit 27 calculates the position of the center of gravity of the capsule endoscope 10 to cause the capsule endoscope 10 to have this posture and calculates rotation angles of the eccentric motors 161 and 162 (see FIGS. 4A and 4B) to realize the position of the center of gravity.

In step S26, the control signal transmitting unit 28 wirelessly transmits information indicating the rotation angles of the eccentric motors 161 and 162 calculated in step S25 to the capsule endoscope 10 as the posture control signal.

In step S14 illustrated in FIG. 6, the control unit 12 of the capsule endoscope 10 determines whether or not the receiving unit 15 has received the posture control signal.

When the receiving unit 15 has received the posture control signal (step S14: Yes), the control unit 12 outputs the posture control signal to the posture changing unit 16 and changes the posture of the capsule endoscope 10 (step S15). Specifically, the control unit 12 changes the rotation angles of the eccentric motors 161 and 162 of the posture changing unit 16, thereby changing the position of the center of gravity of the capsule endoscope 10. In this way, as illustrated in FIG. 11, the posture of the capsule endoscope 10 is changed such that the central portion C in the lumen direction coincides with a center point O of an image m2 corresponding to the center of the visual field V of the capsule endoscope 10.

On the other hand, if the receiving unit 15 has not received the posture control signal (step S14: No), the operation of the capsule endoscope 10 directly proceeds to step S16.

In step S16, the control unit 12 determines whether or not to end the imaging. Specifically, the control unit 12 determines to end the imaging when a predetermined period of time or more has elapsed since the capsule endoscope 10 was turned on, the amount of remaining power of the power source unit 17 becomes smaller than or equal to a predetermined value, or a signal indicating the end of examination is transmitted from the control device 20. When not ending the imaging (step S16: No), the operation of the capsule endoscope 10 returns to step S14. On the other hand, when ending the imaging (step S16: Yes), the capsule endoscope 10 ends the operation.

In step S27 illustrated in FIG. 7, the control device 20 determines whether or not to end the examination using the capsule endoscope 10. Specifically, the control device 20 determines to end the examination when the wireless transmission of the image signal from the capsule endoscope 10 is completed or a user performs an operation to end the examination on the control device 20. When not ending the examination (step S27: No), the operation of the control device 20 returns to step S24. On the other hand, when ending the examination (step S27: Yes), the control device 20 ends the operation. In this case, the control device 20 may transmits a signal indicating the end of examination to the capsule endoscope 10 before ending the operation.

As described above, according to the first embodiment, information indicating the current posture of the capsule endoscope 10 and the posture control signal for changing the posture of the capsule endoscope 10 are transmitted and received through bidirectional communication between the capsule endoscope 10 and the control device 20, and it is possible to cause the capsule endoscope 10 to change the posture of the capsule endoscope 10 based on the posture control signal. Therefore, even when the posture of the capsule endoscope 10 is involuntarily changed by peristaltic action of the subject, it is possible to stabilize the posture of the capsule endoscope 10 by causing the capsule endoscope 10 to change its own posture, which makes it possible to cause the capsule endoscope 10 to continue the imaging with an appropriate visual field.

In the first embodiment described above, the target posture is set such that the center of the visual field V of the capsule endoscope 10 faces the lumen direction. However, the setting method of the target posture is not limited to this. For example, the target posture may be set such that the center of the visual field V faces a characteristic portion of the subject shown in the image displayed on the display unit 23. As a specific example, on the basis of pixel information in the image displayed on the display unit 23, a portion that is estimated to a lesion (for example, a strong red portion) is automatically detected from the image and a direction from the center point of the image to the portion is set as the target posture.

### First Modified Example

Next, a first modified example of the first embodiment of the present invention will be described.

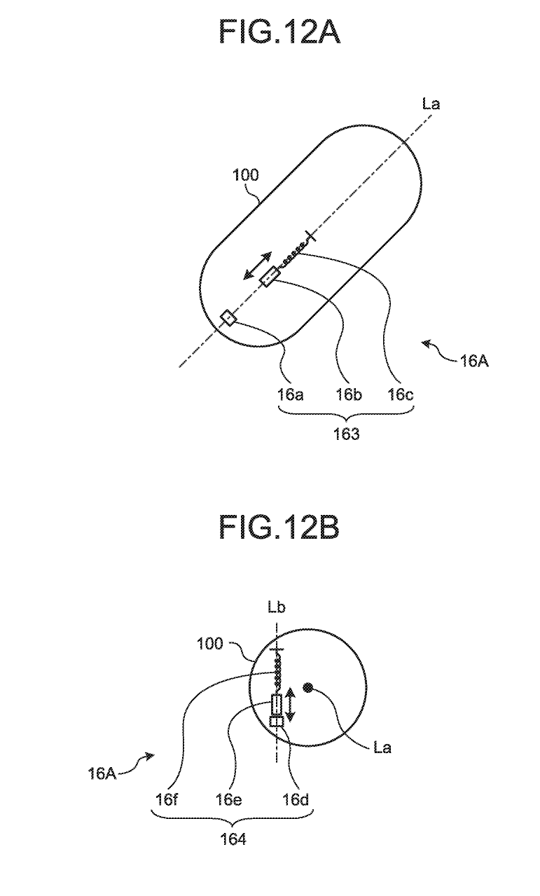

FIGS. 12A and 12B are schematic diagrams illustrating a configuration example of a posture changing unit included in a capsule endoscope according to the first modified example. FIG. 12A illustrates the capsule-shaped casing 100 as viewed from a side surface, and FIG. 12B illustrates the capsule-shaped casing 100 as viewed from a long axis La direction.

A posture changing unit 16A in the first modified example has two gravity center position adjusting units 163 and 164 and a power supply unit (not illustrated in the drawings) that supplies power to the gravity center position adjusting units 163 and 164.

The gravity center position adjusting unit 163 has an electromagnet 16a, a magnetic body 16b formed by a permanent magnet, an iron core, and the like, and a spring 16c whose one end is fixed to a predetermined position in the capsule-shaped casing 100. The magnetic body 16b is connected to the spring 16c and is movable along the long axis La of the capsule-shaped casing 100. When the electromagnet 16a receives power supply and is magnetized, the magnetic body 16b moves to a position determined by a balance between a magnetic force of the electromagnet 16a and an elastic force of the spring 16c. In this way, the position of the center of gravity on the long axis La changes.

The gravity center position adjusting unit 164 has an electromagnet 16d, a magnetic body 16e formed by a permanent magnet, an iron core, and the like, and a spring 16f whose one end is fixed to a predetermined position in the capsule-shaped casing 100. The magnetic body 16e is connected to the spring 16f and is movable along a line Lb on a plane perpendicular to the long axis La of the capsule-shaped casing 100. When the electromagnet 16d receives power supply and is magnetized, the magnetic body 16e moves to a position determined by a balance between a magnetic force of the electromagnet 16d and an elastic force of the spring 16f. In this way, the position of the center of gravity on the line Lb changes.

In the first modified example, the posture changing unit 16A changes the posture (elevation angle θ, turning angle ψ) of the capsule endoscope 10 by adjusting the power supplied to the electromagnets 16a and 16d according to the posture control signal wirelessly transmitted from the control device 20 and changing the position of the center of gravity of the capsule endoscope 10.

### Second Modified Example

Next, a second modified example of the first embodiment of the present invention will be described.

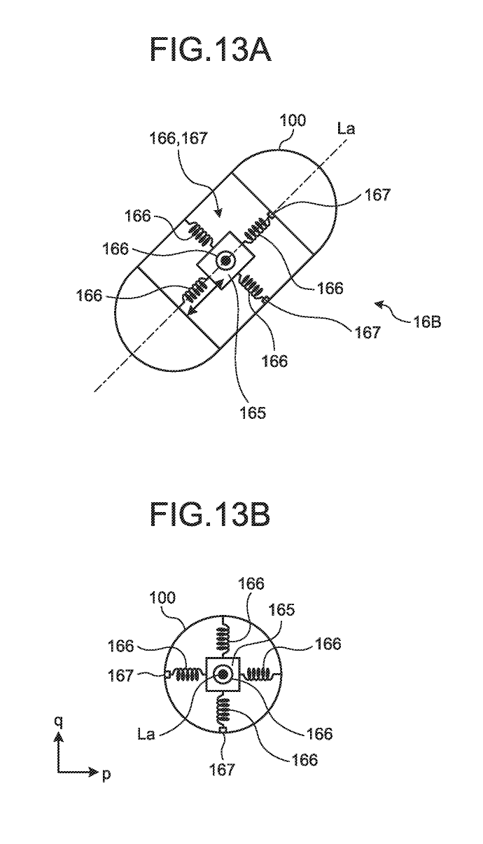

FIGS. 13A and 13B are schematic diagrams illustrating a configuration example of a posture changing unit included in a capsule endoscope according to the second modified example. FIG. 13A illustrates the capsule-shaped casing 100 as viewed from a side surface, and FIG. 13B illustrates the capsule-shaped casing 100 as viewed from a long axis La direction.

A posture changing unit 16B in the second modified example has a weight 165 attached inside the capsule-shaped casing 100 through six springs 166, three spring winding units 167, a winding drive unit (not illustrated in the drawings) that drives the spring winding units 167. The weight 165 is provided movably in three directions, which include a direction of the long axis La of the capsule-shaped casing 100 and two directions (p direction and q direction) perpendicular to each other on a plane perpendicular to the long axis La, by the six springs 166. The three spring winding units 167 are provided in the direction of the long axis La, the p direction, and the q direction, respectively.

In the second modified example, the posture changing unit 16B changes the position of the weight 165 by adjusting the amount of winding of each spring 166 wound by the spring winding unit 167 according to the posture control signal wirelessly transmitted from the control device. Hence, the position of the center of gravity of the capsule endoscope 10 is changed, thereby changing the posture (elevation angle θ, turning angle ψ) of the capsule endoscope 10.

### Second Embodiment

Next, a second embodiment of the present invention will be described.

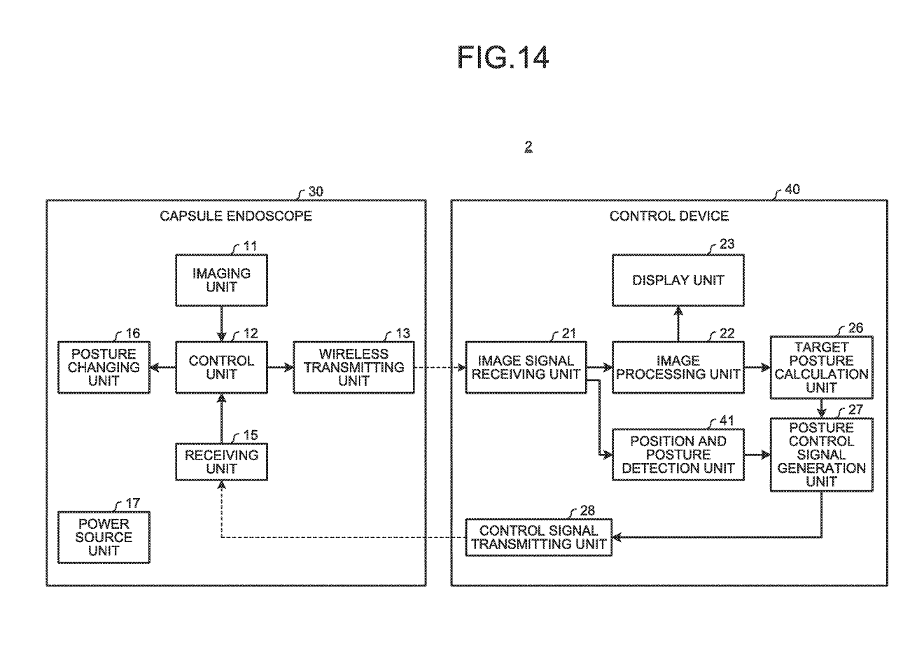

FIG. 14 is a block diagram illustrating a configuration of a capsule endoscope system according to the second embodiment of the present invention. As illustrated in FIG. 14, a capsule endoscope system 2 according to the second embodiment includes a capsule endoscope 30 and a control device 40.

Although operations of elements of the capsule endoscope 30 illustrated in FIG. 14 are the same as those of the first embodiment, the capsule endoscope 30 is not provided with the position and posture information sending unit 14 that generates a magnetic field, which is provided in the capsule endoscope 10 illustrated in FIG. 2. In the second embodiment, the wireless signal (image signal) transmitted by the wireless transmitting unit 13 is used as information related to the position and posture of the capsule endoscope 30. In other words, the wireless transmitting unit 13 has a function of the position and posture information sending unit.

The control device 40 includes a position and posture detection unit 41 instead of the position and posture information acquisition unit 24 and the position and posture detection unit 25 illustrated in FIG. 1. In the second embodiment, the image signal receiving unit 21 that receives the wireless signal transmitted by the capsule endoscope 30 has a function of a position and posture information acquisition unit that acquires information related to the position and posture of the capsule endoscope 30.

The position and posture detection unit 41 receives wireless signals through a plurality of receiving antennas included in the image signal receiving unit 21 and detects the position and posture of the capsule endoscope 30 based on the intensity distribution of the wireless signals. As a detection method of the position and posture of the capsule endoscope 30, it is possible to use various known methods. As an example, it is possible to obtain the position and posture of the capsule endoscope 30 by appropriately setting an initial value of the position of the capsule endoscope 30 and repeating processing of calculating an estimated value of the position and posture by using the Gauss-Newton method until a difference between the calculated estimated value and the previous estimated value becomes smaller than or equal to a predetermined value (for example, see JP 2007-283001 A). The configuration and operation of each unit other than the position and posture detection unit 41 in the control device 40 are the same as those in the first embodiment.

As described above, according to the second embodiment of the present invention, since the wireless signal transmitted from the capsule endoscope 30 is used as information related to the position and posture, it is not necessary to provide a dedicated configuration (such as the resonance circuit including the coil 141 and the capacitor 142 illustrated in FIG. 3 and the sensing coil 24a illustrated in FIG. 2) for detecting the position and posture of the capsule endoscope 30. Therefore, it is possible to simplify the configuration of the capsule endoscope 30 and the control device 40.

### Third Embodiment

Next, a third embodiment of the present invention will be described.

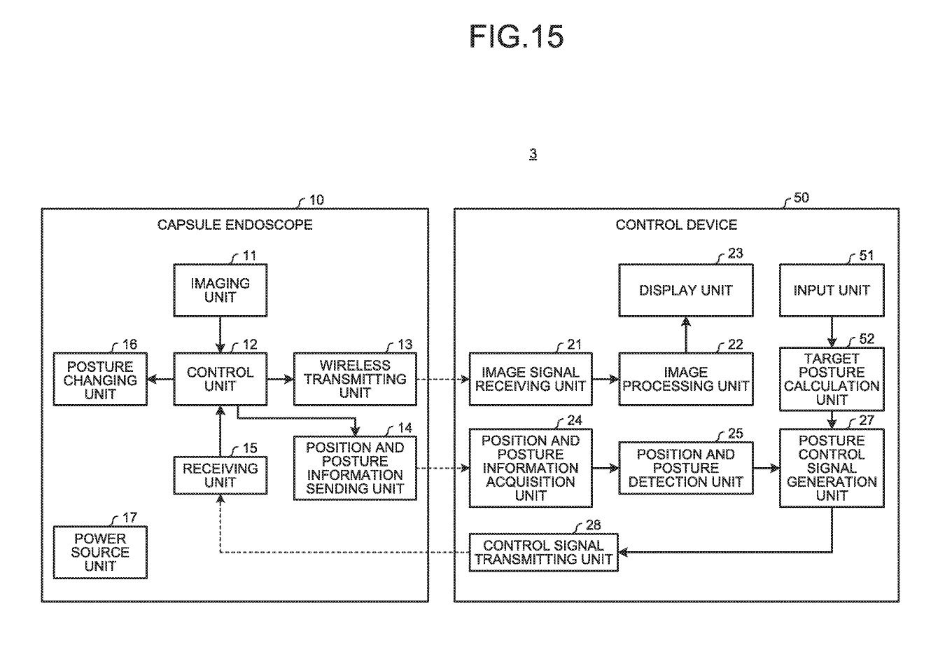

FIG. 15 is a block diagram illustrating a configuration of a capsule endoscope system according to the third embodiment of the present invention. As illustrated in FIG. 15, a capsule endoscope system 3 according to the third embodiment includes the capsule endoscope 10 and a control device 50. The configuration and operation of the capsule endoscope 10 are the same as those in the first embodiment.

The control device 50 includes an input unit 51, which is used when a user inputs various commands and information, in addition to the elements of the control device 20 illustrated in FIG. 1, and a target posture calculation unit 52 instead of the target posture calculation unit 26 illustrated in FIG. 1. The input unit 51 includes input devices such as a keyboard and a mouse and an operation console having various buttons and various switches. The input unit 51 inputs a signal according to an operation performed by a user from the outside into the target posture calculation unit 52. The target posture calculation unit 52 calculates the target posture of the capsule endoscope 10 based on the signal input from the input unit 51.

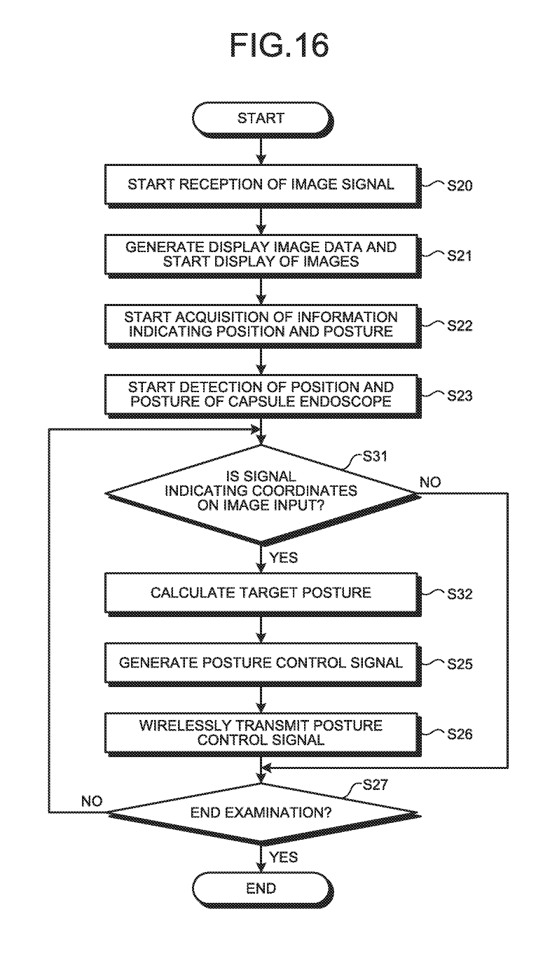

Next, an operation of the capsule endoscope system 3 will be described with reference to FIG. 16. FIG. 16 is a flowchart illustrating an operation of the control device 50. The operation of the capsule endoscope 10 is the same as that of the first embodiment (see FIG. 6). Steps S20 to S23 in FIG. 16 are the same as those in the first embodiment.

After step S21, the display unit 23 of the control device 50 displays images of inside of the subject based on image signals sequentially wirelessly transmitted from the capsule endoscope 10. A user observes these images and when the user wants to change a direction of the visual field of the capsule endoscope 10, the user specifies a portion in an image, which the user wants to move to the center of the image, by using the input unit 51. For example, as illustrated in FIG. 9, when the user wishes to move the central portion C in the lumen direction shown in the image m1 to the center point O, the user specifies the central portion C by using the input unit 51. Specifically, the user double-clicks the central portion C by using a mouse or encloses the central portion C by using a cursor on the screen of the display unit 23. The input unit 51 inputs a signal indicating coordinates specified on the screen of the display unit 23 into the target posture calculation unit 52.

In step S31 that follows step S23, the target posture calculation unit 52 determines whether or not a signal indicating coordinates on an image displayed on the display unit 23 is input from the input unit 51.

When the signal indicating coordinates on the image is input (step S31: Yes), the target posture calculation unit 52 calculates the target posture of the capsule endoscope 10 (step S32). Specifically, the target posture calculation unit 52 calculates a directional vector directed from a center point of the image to the coordinates input in step S31 and calculates the target posture based on the directional vector.

For example, when the coordinates of the central portion C in the lumen direction illustrated in FIG. 9 is input, the target posture calculation unit 52 first calculates a directional vector v directed from the center point O of the image m1 to the central portion C. Then, the target posture calculation unit 52 calculates a posture, where the center of the visual field V of the capsule endoscope 10 faces a target point, as the target posture of the capsule endoscope 10, from the length (the number of pixels in the image m1) and the orientation of the directional vector v. The next step S25 and the following steps are the same as those in the first embodiment.

If the signal indicating coordinates is not input (step S31: No), the operation of the control device 50 proceeds to step S27.

As described above, according to the third embodiment of the present invention, it is possible to cause the capsule endoscope 10 to change the posture of the capsule endoscope 10 such that the visual field of the capsule endoscope 10 faces a portion desired by a user.

In the first to the third embodiment described above, the exemplary capsule endoscope has been described which is configured to be orally introduced into the subject and captures images a lumen of the subject. However, the present invention is not limited by these embodiments. It is possible to apply the present invention to various endoscopes which have a capsule shape and which are introduced into a subject and capture images.

The first to the third embodiments and the modified examples of the embodiments described above are merely examples for implementing the present invention, and the present invention is not limited to these embodiments and modified examples. The present invention can form various inventions by appropriately combining a plurality of elements disclosed in the first to the third embodiments and the modified examples. From the above description, it is obvious that the present invention can be variously modified according to specifications and the like, and further, other various embodiments are possible within the scope of the present invention.

According to some embodiments, the information indicating the posture of the capsule endoscope and the control signal to change the posture of the capsule endoscope are transmitted and received through bidirectional communication between the capsule endoscope and the control device and the posture changing unit that changes the posture of the capsule endoscope based on the control signal is provided in the capsule endoscope. With this feature, even after the capsule endoscope is introduced into the subject, the capsule endoscope can change the posture by itself.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A capsule endoscope system comprising:
a capsule endoscope configured to be introduced into a subject, configured to perform imaging to generate an image signal, and configured to wirelessly transmit the image signal; and
a control device configured to receive the image signal to generate an image of an inside of the subject based on the image signal,
wherein the capsule endoscope comprises:
a first transmitter configured to send information indicating a posture of the capsule endoscope;
a first receiver configured to receive a signal transmitted from the control device; and
a posture changing mechanism configured to change the posture of the capsule endoscope,
wherein the control device comprises:
a second receiver configured to acquire the information indicating the posture sent by the first transmitter;
a posture detector configured to detect the posture of the capsule endoscope based on the information indicating the posture;
a target posture calculator configured to calculate a target posture of the capsule endoscope based on the image;
a posture control signal generator configured to generate a control signal for changing the posture of the capsule endoscope based on a detection result of the posture of the capsule endoscope and based on the target posture; and
a second transmitter configured to transmit the control signal to the capsule endoscope,
wherein the first receiver is configured to receive the control signal transmitted by the second transmitter, and
the posture changing mechanism is configured to change the posture of the capsule endoscope based on the control signal received by the first receiver.

2. The capsule endoscope system according to claim 1, wherein
the first transmitter comprises a coil configured to receive power supply to generate a magnetic field,
the second receiver comprises a plurality of coils configured to detect the magnetic field to output a plurality of detection signals, respectively, and
the posture detector is configured to detect the posture of the capsule endoscope based on the plurality of detection signals respectively output by the plurality of coils.

3. The capsule endoscope system according to claim 1, wherein
the first transmitter is configured to transmit the image through a radio wave,
the second receiver comprises a plurality of antennas configured to receive the radio wave, and
the posture detector is configured to detect the posture of the capsule endoscope based on intensity of the radio wave received by each of the plurality of antennas.

4. The capsule endoscope system according to claim 1, wherein
the posture changing mechanism is configured to change the posture of the capsule endoscope by changing a position of a center of gravity of the capsule endoscope.

5. The capsule endoscope system according to claim 1, wherein
the target posture calculator is configured to calculate the target posture such that a specific portion of the subject shown in the image coincides with a center of an imaging visual field of the capsule endoscope.

6. The capsule endoscope system according to claim 1, further comprising an input unit configured to input a signal in response to an operation from outside into the target posture calculator,
wherein when an input for specifying a specific portion in the image is input into the target posture calculator from the input unit, the target posture calculator is configured to calculate the target posture such that a position of the subject corresponding to the specified specific portion coincides with a center of an imaging visual field of the capsule endoscope.
